(19) United States
(12) Patent Application Publication
(10) Pub. No.: US 2023/0190793 A1
(43) Pub. Date: Jun. 22, 2023

(54) Pharmaceutical Preparation for Use in the Treatment of Systemic Inflammatory Response Syndrome (SIRS)

(71) Applicant: Cytopharma GmbH, Darmstadt (DE)

(72) Inventors: Dirk Kaiser, Eppertshausen (DE); Mark Andre Freyberg, Freiburg (DE)

(21) Appl. No.: 17/925,851

(22) PCT Filed: May 11, 2021
(86) PCT No.: PCT/EP2021/062480

§ 371 Date: Nov. 17, 2022

(30) Foreign Application Priority Data
May 18, 2020 (DE) 10 2020 113 383.9

(51) Int. Cl. A61K 33/40; A61K 33/20

(52) U.S. Cl. CPC A61K 33/40; A61K 33/20

(57) ABSTRACT

The present invention describes a pharmaceutical preparation for use in the treatment of systemic inflammatory response syndrome (SIRS).

The present invention relates to a pharmaceutical preparation for use in the treatment of systemic inflammatory response syndrome (SIRS).

Systemic inflammatory response syndrome (SIRS) is an inflammatory reaction of the human organism that is difficult to treat. Among adult patients registered in an emergency department (ED) in the USA, approximately 17.8% were diagnosed with SIRS; 26% of them had an infection (Horeczko, T., et al. (2014). “Epidemiology of the Systemic Inflammatory Response Syndrome (SIRS) in the emergency department.” West J Emerg Med 15(3): 329-336). A study of SIRS patients, 41% of whom had a diagnosis of sepsis, showed a mortality rate of 24% after 30 days (Schrijver, I. T., H. et al. (2017). “Myeloperoxidase can differentiate between sepsis and non-infectious SIRS and predicts mortality in intensive care patients with SIRS.” Intensive Care Med Exp 5(1): 43). The incidence of sepsis increased to 240.4 per 100,000 people in the USA from 1979 to 2000, the mortality rate from sepsis was 17.9% from 1995 to 2000 (Martin, G. S., et al. (2003). “The epidemiology of sepsis in the United States from 1979 through 2000.” N Engl J Med 348(16): 1546-1554).

The exact causes of SIRS have not been definitively determined, such that, at present, substantially only symptoms, such as hypotension or hypertension or body temperature that is too low or too high, etc., are treated by intensive care medical methods. Depending on the physical condition of the patient, they either improve or die, often due to multiple organ failure.

In 2002 the drug drotrecogin was approved for the treatment of patients with severe sepsis who had a very low chance of survival (Warren H S, et al. (2002). “Risks and benefits of activated protein C treatment for severe sepsis”. N. Engl. J. Med. 347 (13): 1027-30). Drotrecogin is a recombinant form of activated protein C found in the blood and has been marketed by Eli Lilly under the brand name Xigris®. However, it caused severe side effects, and studies published in 2011 showed that for patients with sepsis there was no significant survival advantage from taking Xigris®. Therefore, the sale of Xigris® was discontinued in 2011.

To date, no drugs have arisen allowing for the successful treatment of SIRS. Blocking the cytokines typical of SIRS appears to be one option for controlling an excessive inflammatory reaction. However, no therapies are yet available in which antibodies are used against individual cytokines for example; only the natural Activated Protein C (APC) can be used as an anti-inflammatory protein that inhibits blood clotting (Cao, C., et al. (2010). “The efficacy of activated protein C in murine endotoxemia is dependent on integrin CD11 b.” J Clin Invest 120(6): 1971-1980; Donati, A., et al. (2013). “The aPC treatment improves microcirculation in severe sepsis/septic shock syndrome.” BMC Anesthesiol 13(1): 25). Therapeutic measures for infection are usually taken in SIRS after the positive diagnosis of an infection in the blood culture (sepsis); the choice of appropriate antibiotic agent is then tailored to the pathogen. In a critical phase of SIRS, problems relating to the cardiovascular system, such as edema or a drop in blood pressure are responded to by supplying fluids or by raising the blood pressure with medication.

In light of the prior art, it is therefore the objective of the present invention to provide a pharmaceutical preparation for use in the treatment of systemic inflammatory response syndrome (SIRS). In this regard, the pharmaceutical preparation should be as simple and inexpensive to produce as possible. In particular, the pharmaceutical preparation should be able to be produced in large quantities. Furthermore, the pharmaceutical preparation should be very well tolerated and have as few side effects as possible. Furthermore, the pharmaceutical preparation should be highly effective. In particular, it should be possible to administer the pharmaceutical preparation at the first signs of SIRS without causing unacceptable harm to the patient. In addition, the pharmaceutical preparation should have such high efficacy that even patients with a severe form SIRS will have a significantly higher chance of survival after the administration of the drug.

Furthermore, an objective of the present intention is to provide a pharmaceutical preparation which can be handled safely and easily.

Furthermore, the pharmaceutical preparation should be easily adaptable to specifications relating to the dosage, for example so that a relatively high dosage can be applied. In addition, it is also an objective of the present invention to provide a pharmaceutical preparation which has excellent tolerability, in particular has few side effects. Furthermore, the pharmaceutical preparation should contribute to an improvement in quality of life and not additionally impair it.

Furthermore, the pharmaceutical preparation to be produced should lead to a significant improvement in the survival rate of patients with SIRS, particularly in the case of severe SIRS.

These objectives and further objectives, which are not explicitly mentioned, but can be easily derived or developed from the discussion in the introduction, are achieved by a pharmaceutical preparation having all of the features of claim 1.

Thus, the subject-matter of the present invention is to provide a pharmaceutical preparation for use in the treatment of systemic inflammatory response syndrome (SIRS), containing a reactive chlorine compound as active ingredient.

In particular, by means of the present invention it is possible to treat systemic inflammatory response syndrome (SIRS) well, reliably, safely and with few side effects.

In particular, a pharmaceutical preparation according to the present invention for use in the treatment of systemic inflammatory response syndrome (SIRS) enables a significant increase in the survival rate of patients with SIRS, particularly those with severe SIRS.

In addition, a pharmaceutical preparation according to the invention shows excellent tolerability, in particular has few side effects. Furthermore, a pharmaceutical preparation according to the present invention leads to an improvement in quality of life and does not additionally impair it.

Furthermore, a pharmaceutical preparation according to the invention is very well tolerated and has relatively few side effects. On the other hand, a pharmaceutical preparation according to the invention has a high degree of efficacy, wherein the pharmaceutical preparation can be administered at the first signs of SIRS without causing unacceptable harm to the patients. Furthermore, a pharmaceutical preparation according to the present invention has such high efficacy that even patients with a severe form of SIRS experience a significant increase their chances of survival after administration of the drug.

Preferably, the pharmaceutical preparation can be produced relatively easily and inexpensively. Furthermore, a pharmaceutical preparation can be produced in large quantities.

Furthermore, the pharmaceutical preparation can be handled safely and easily.

Furthermore, the pharmaceutical preparation can easily be adapted to specifications relating to dosage, which means that relatively high or low dosages can be used for example, so that they can be adjusted to the respective needs of the patients.

The pharmaceutical preparation, containing a reactive chlorine compound as active ingredient, is used for the treatment of systemic inflammatory response syndrome (SIRS).

The term SIRS (systemic inflammatory response syndrome) is well known and was defined at a conference on the subject of sepsis and organ failure in 1991 (Bone, R. C., et al. (1992). “Definitions for sepsis and organ failure and guidelines for the use of innovative therapies in sepsis. The ACCP/SCCM Consensus Conference Committee. American College of Chest Physicians/Society of Critical Care Medicine.” Chest 101(6): 1644-1655).

There are various triggers for SIRS, such as infections with bacteria, fungi or injuries, though what they all have in common is that a sequence of reactions occurs in the body which lead to a life-threatening condition with multiple-organ failure and shock (drop in blood pressure). The term sepsis was coined by Schottmüller for the variant of this disease caused by infections, triggered by bacteria, viruses, fungi or parasites, several years after Pasteur first detected bacteria in the blood of sick people in 1880 (Annane, D., et al. (2005). “Septic shock.” Lancet 365(9453): 63-78).

The groups of triggers mentioned above, infectious triggers on the one hand and other organ damage and injuries, such as surgery, trauma, burns or inflammation such as pancreatitis on the other hand, substantially lead to an identical progression of the disease and to similar symptoms.

Systemic inflammatory response syndrome (SIRS) is subdivided into diseases that are not caused by infection and those with an underlying infection known as sepsis which is part of the overall SIRS complex. The infections spreading in the blood can be caused by bacteria, fungi, parasites, viruses and other pathogens (Bone 1992).

SIRS is often diagnosed when at least two of the following four criteria are met:
body temperature is either elevated (>38° C.) or depressed (<36° C.)
tachycardia (heart rate >90/min)
tachypnoea (respiratory rate >20/min) or arterial carbon dioxide partial pressure PaCO2<33 mm Hg (Torr); equivalent to <4.3 kPa
leukocytosis (leukocytes >12,000/μL) or leukopenia (<4,000/μL).

These diagnostic criteria were proposed by Levy (Levy, M. M., et al. (2003). “2001 SCCM/ESICM/ACCP/ATS/SIS International Sepsis Definitions Conference.” Crit Care Med 31(4): 1250-1256).

The diagnostic criteria for SIRS are supported by molecular analyses, where a number of cytokines and other marker proteins were found in altered concentrations (Reichsoellner, M., et al. (2014). “Clinical evaluation of multiple inflammation biomarkers for diagnosis and prognosis for patients with systemic inflammatory response syndrome.” J Clin Microbiol 52(11): 4063-4066; Ueda, S., K. Nishio, N. Minamino, A. Kubo, Y. Akai, K. Kangawa, H. Matsuo, Y. Fujimura, A. Yoshioka, K. Masui, N. Doi, Y. Murao and S. Miyamoto (1999). “Increased plasma levels of adrenomedullin in patients with systemic inflammatory response syndrome.” Am J Respir Crit Care Med 160(1): 132-136). Some of these markers can be used diagnostically when it is necessary in SIRS patients to decide quickly between an infectious (sepsis) and non-infectious SIRS. Thus the increased concentration in the blood of interleukin 6 (IL-6), C-reactive protein (CRP), procalcitonin (PCT) or lipopolysaccharide binding protein (LBP) could indicate the presence of infectious pathogens much earlier than an elaborate test using blood cultures (Meynaar, I. A., et al. (2011). “In Critically III Patients, Serum Procalcitonin Is More Useful in Differentiating between Sepsis and SIRS than CRP, 11-6, or LBP.” Crit Care Res Pract 2011: 594645; Reichsoellner 2014). If four SIRS criteria are met, a bacterial infection of the bloodstream is probable (Grozdanovski, K., et al. (2019). “Association of Systemic Inflammatory Response Syndrome with Bacteremia in Patients with Sepsis.” Pril (Makedon Akad Nauk Umet Odd Med Nauki) 40(2): 51-56).

In the case of an exacerbation of the disease with existing organ damage, for example shown by the SOFA score (SOFA: Sequential Organ Failure Assessment), several parameters are determined on the function of different organs as well as on blood pressure and blood coagulation and combined into one assessment score. If at least one organ is damaged, it used to be called “severe sepsis”, but this term should no longer be used according to the recommendations of the third Consensus Conference in 2015 (Singer, M., C. S. et al. (2016). “The Third International Consensus Definitions for Sepsis and Septic Shock (Sepsis-3).” Jama 315(8): 801-810). It should be noted that the SOFA score criteria do not include infection data, but reflect the organ functionality. The same data are also used to assess the severity of SIRS without an infectious cause. The SOFA score is mainly used to assess the progression of the disease in patients in an intensive care unit, while the qSOFA score (quick SOFA) enables rapid assessment of the patient in an outpatient or normal ward of a clinic (Singer 2016).

The assessment of a deterioration in health status by the SOFA score is based on analysis of the function of the organs: lungs, kidneys, liver, central nervous system (CNS) as well as blood coagulation. A further increase in the critical state leads to so-called septic shock, where hypotension is the decisive criterion (Annane, D., et al. (2005). “Septic shock.” Lancet 365(9453): 63-78; Singer 2016) with mortality rates of up to 80% (Jawad I, et al. (2012). “Assessing available information on the burden of sepsis: global estimates of incidence, prevalence and mortality” Journal of global health 2: 1, doi: 10.7189/jogh.02.010404). To detect an infection usually a blood culture is taken to detect infections caused by bacteria (gram negative or gram positive) or by fungi in the blood. However, for patients with organ damage or shock, an infection need not be present, but in the clinical picture of SIRS the impaired function of organs or the cardiovascular system can also be triggered by non-infectious damage, presumably by the increased release of cytokines (Fujishima, S. (2016). “Organ dysfunction as a new standard for defining sepsis.” Inflamm Regen 36: 24).

The high number of negative results from blood cultures (without infection) in SIRS/sepsis patients at over 50% [Examples: Sepsis, 53.4% (Guo, S. Y. et al. (2015) “Procalcitonin Is a Marker of Gram-Negative Bacteremia in Patients With Sepsis” Am. J. Med. Sci. 349: 6 499-504); SIRS, 79.2% (Grozdanovski 2019)] indicates that patients can be divided into three groups: (a) infected patients; (b) patients no longer infected at the time of blood culture, where cell wall components of the bacteria may still be active after treatment with antibiotics (Hurley J. C., et al. (1991) “Antibiotic-Induced Release of Endotoxin in Chronically Bacteriuric Patients” Antimicrob. Agents Chemother 35: 11); (c) patients, who were not infected but where injury, trauma and other events have resulted in organ damage (Matsuda, N., Hattori Y., (2006) “Systemic Inflammatory Response Syndrome (SIRS): Molecular Pathophysiology and Gene Therapy” J Pharmacol Sci 101, 189-198).

Both non-infectious damage and infection can in many cases lead to severe inflammatory reactions, including organ damage and shock. The triggers are not yet fully understood, but it is assumed that similar cascades occur for both reasons.

In the case of an infection with bacteria, the release of lipopolysaccharide (LPS, “endotoxin”) or peptidoglycan from the bacterial membrane can stimulate an immune response via signaling receptors of the Toll-like receptors (TLR) family, such as TLR4, or dimers TLR1/TLR2 and TLR6/TLR2 (Kopp, E., R. Medzhitov (2003). “Recognition of microbial infection by Toll-like receptors.” Curr Opin Immunol 15(4): 396-401).

The general principle is that molecules of an infectious pathogen, known as PAMPs (Pathogen-Associated Molecular Patterns), such as LPS and peptidoglycan, but also nucleic acids (DNA, RNA), bind to one of several receptors, known as PRRs (Pattern Recognition Receptors, such as for example TLRs). This triggers a signal cascade which leads to the synthesis and release of cytokines as part of an immune reaction (Akira, S., et al. (2006). “Pathogen recognition and innate immunity.” Cell 124(4): 783-801). In addition to the membrane-bound TLRs there are other PRRs, such as NOD-like receptors NOD1 and NLRP3, which are located in the cytoplasm of the cell (Kim, Y. K., et al. (2016). “NOD-Like Receptors in Infection, Immunity, and Diseases.” Yonsei Med J 57(1): 5-14).

When tissue structures are damaged by injury or surgery, generally referred to as “trauma”, unplanned cell death, referred to as necrosis, results in the release of active cell components, known as DAMPs (danger-associated molecular patterns) or alarmin. Similar to PAMPs, these also bind to receptors and trigger inflammation-promoting signals (Hirsiger, S., et al. (2012). “Danger signals activating the immune response after trauma.” Mediators Inflamm 2012:315941; Hwang, P. F., et al. (2011). “Trauma is danger.” J Transl Med 9: 92). Since some of the DAMP molecules, such as the proteins HMGB1 and Hsp70 and mitochondrial DNA, bind to PRRs (various TLRs, NLRP3) in a similar manner to the PAMPs of infectious agents (Brenner, C., et al. (2013). “Decoding cell death signals in liver inflammation.” J Hepatol 59(3): 583-594; Yuan, X., et al. (2019). “Protective Effect of Hesperidin Against Sepsis-Induced Lung Injury by Inducing the Heat-Stable Protein 70 (Hsp70)/Toll-Like Receptor 4 (TLR4)/Myeloid Differentiation Primary Response 88 (MyD88) Pathway.” Med Sci Monit 25: 107-114), a very similar pattern of cytokines is observed in response to trauma as in bacterial infection (Bianchi, M. E. (2007). “DAMPs, PAMPs and alarmins: all we need to know about danger.” J Leukoc Biol 81(1): 1-5; Brenner 2013; Hirsiger 2012; Lorne, E., et al. (2010). “Toll-like receptors 2 and 4: initiators of non-septic inflammation in critical care medicine?” Intensive Care Med 36(11): 1826-1835).

Therefore, the general concept derived from the professional community is that so far SIRS is characterized by alarm signals, which are released in the form of PAMPs during an infection or in the form of DAMPs during non-infectious events such as trauma and then activate the same signaling pathways via PRRs. This explains the strong inflammatory response observed in SIRS in the form of released cytokines, such as IL-1, IL-6, TNF alpha or interferon gamma (IFN gamma) (Hirsiger 2012; Matera, G., et al. (2013). “Impact of interleukin-10, soluble CD25 and interferon-gamma on the prognosis and early diagnosis of bacteremic systemic inflammatory response syndrome: a prospective observational study.” Crit Care 17(2): R64; Solomkin, J. S., et al. (1994). “Alterations of neutrophil responses to tumor necrosis factor alpha and interleukin-8 following human endotoxemia.” Infect Immun 62(3): 943-947; Volpin, G., et al. (2014). “Cytokine levels (IL-4, IL-6, IL-8 and TGFbeta) as potential biomarkers of systemic inflammatory response in trauma patients.” Int Orthop 38(6): 1303-1309).

Until now it has been assumed by the professional community that generally in SIRS the strong immune reaction triggered by cytokines, such as for example TNF alpha or IL-6, eventually leads to organ damage and death in many patients. The concentration of cytokines IL-6, IL-7 and IL-10 and chemokines MCP-1 and IL-8 in the blood of sepsis patients indeed seems to correlate with the risk of not surviving (Hong, T. H., et al. (2014). “Biomarkers of early sepsis may be correlated with outcome.” J Transl Med 12: 146). In SIRS without infection the cytokines IL-6, IL-12 and IL-1 beta and TNF alpha and chemokines IL-8 are found to be elevated (Volpin 2014). Accordingly, animal models that produce SIRS via infection have a general significance with regard to SIRS.

The released cytokines in SIRS have a range of effects on endothelial cells, on immune cells and on blood coagulation. Thus for example, the cytokines TNF alpha and IL-1 beta were able to trigger shock-like reactions in animal experiments, such as in septic shock, although no infection was present (Fujishima 2016). The released cytokines also lead to the increased formation of adhesion proteins on endothelial cells, which enables the attachment and migration of immune cells into the affected tissue. Furthermore, the blood vessels may dilate and the blood pressure may drop. Due to the change in form (contraction) of individual endothelial cells or cell damage, the permeability to blood components may increase and the ability of endothelial cells to act as blood coagulants may decrease (King, E. G., et al. (2014). “Pathophysiologic mechanisms in septic shock.” Lab Invest 94(1): 4-12). Therapeutics steps, such as increasing blood pressure or giving fluids, are aimed at restoring blood flow or are aimed at fighting an infection.

Triggered by the effect of individual cytokines, SIRS causes a shift in the balance between procoagulant and anticoagulant factors, such that increased blood clotting, together with reduced blood flow, occurs in small vessels in particular, and severely impairs the supply to entire organs, such as the lungs or kidneys (Petäjä, J. (2011) “Inflammation and coagulation. An overview” Thrombosis Research 127 S34-S37; Rittirsch, D., et al. (2008). “Harmful molecular mechanisms in sepsis.” Nat Rev Immunol 8(10): 776-787). Here the clinical finding of disseminated intravascular coagulation (DIG) is used, which may be responsible for organ damage in the course of SIRS. The increased coagulation can subsequently lead to a deficiency of clotting factors and platelets, so that in SIRS and sepsis with increased coagulation, bleeding may also occur afterwards (Levi, M., van der Poll, T., (2010). “Inflammation and coagulation.” Crit Care Med 38(2 Suppl): S26-34).

Lung function is often impaired with SIRS, which is also attributed to damage to the endothelial cells in the capillaries of the lungs. As a result, there is increased permeability of the capillaries, wherein the penetration of fluid, immune cells and proteins from the blood into the alveoli massively impairs the gas exchange in the lungs and results in death rates of around 40% (Hukkanen, R. R., et al. (2009). “Systemic inflammatory response syndrome in nonhuman primates culminating in multiple organ failure, acute lung injury, and disseminated intravascular coagulation.” Toxicol Pathol 37(6): 799-804; Tsushima, K., et al. (2009). “Acute lung injury review.” Intern Med 48(9): 621-630).

A immune reaction generally triggered by infections requires two parts, the natural immune system, which is firstly active with granulocytes, monocytes and macrophages, and the adaptive immune system which reacts with activated T-cells and B-cells, which then release antibodies as mature plasma cells. In infections caused by bacteria and fungi, neutrophil granulocytes (“neutrophils”) are significant, which very quickly take in the invading pathogens, for example by means of phagocytosis, and fight them with reaction oxygen species (ROS) or with the enzyme lysozyme (Hajdamowicz, N. H., et al. (2019). “The Impact of Hypoxia on the Host-Pathogen Interaction between Neutrophils and Staphylococcus aureus.” Int J Mol Sci 20(22); Mocsai, A. (2013). “Diverse novel functions of neutrophils in immunity, inflammation, and beyond.” J Exp Med 210(7): 1283-1299). A further process which is performed by neutrophils, known as NETosis, results in the release of chromosomal DNA with the purpose of preventing the spread of bacteria by trapping them in a DNA net (“NET”) (Giacalone, V. D., et al. (2020). “Neutrophil Adaptations upon Recruitment to the Lung: New Concepts and Implications for Homeostasis and Disease.” Int J Mol Sci 21(3); Yipp, B. G., et al. (2012). “Infection-induced NETosis is a dynamic process involving neutrophil multitasking in vivo.” Nat Med 18(9): 1386-1393).

The high concentration of IL-8 in SIRS without infection and in sepsis attracts many neutrophils and could explain organ damage by substances released from the cells, such as hydrogen peroxide, hypochlorite and others (Hong 2014; Mocsai 2013; Solomkin 1994; Volpin 2014). Prolonged activity of the neutrophils is normally limited by cell death in the form of apoptosis. On the other hand in SIRS the apoptosis of lymphocytes (T-cells, B-cells, NK-cells) and granulocytes can severely reduce the capacity of the immune system and thus cause late infections (Hotchkiss, R. S., et al. (2005) “Accelerated Lymphocyte Death in sepsis Occurs by both the Death Receptor and Mitochondrial Pathways” J Immunol 174:5110-5118, doi: 10.4049/jimmunol.174.8.5110; King 2014; Rittirsch 2008; Torre, D., et al. (2003). “Circulating levels of FAS/APO-1 in patients with the systemic inflammatory response syndrome.” Diagn Microbiol Infect Dis 45(4): 233-236).

For example, animal models for SIRS use LPS, which is injected into mice together with other substances and leads to increased concentrations of cytokines, although there is no infection (Bhargava, R., (2013). “Acute lung injury and acute kidney injury are established by four hours in experimental sepsis and are improved with pre, but not post, sepsis administration of TNF-alpha antibodies.” PLoS One 8(11): e79037; Cao (2010). A second method is cecal ligation and puncture (CLP) in mice or rats, wherein material from the cecum leaks into the abdomen, causing SIRS (Hubbard, W. J., et al. (2005). “Cecal ligation and puncture.” Shock 24 Suppl 1: 52-57). For example, the CLP model causes particularly severe damage to the kidneys and lungs (Bhargava 2013), two organs, that are also frequently affected by organ failure in severe SIRS (Fujishima 2016; Hukkanen 2009), so that this model is more suitable for testing new forms of therapy than the LPS model.

In general, for the treatment of SIRS, patients in an intensive care unit are kept under close observation so that immediate intervention can take place if critical parameters change. The main problems associated with SIRS are organ failure and shock. In the presence of an infection, where SIRS is often referred to as sepsis, a blood culture is used to decide whether additional antibiotic measures need to be taken and if so which measures. The SOFA score has become established as a diagnostic criterion for the severity of the disease (SIRS including sepsis), which evaluates the function of several organs, including the CNS, as well as the efficiency of blood circulation (Fujishima 2016).

The pharmaceutical preparation for use in the treatment of systemic inflammatory response syndrome (SIRS) of the present invention comprises at least one reactive chlorine compound as an active ingredient. In this way the pharmaceutical preparation of the present invention can include exactly one, two, three, four or more reactive chlorine compounds. Reactive chlorine compounds are compounds which contribute to an improvement of systemic inflammatory response syndrome (SIRS). It can be assumed that reactive chlorine compounds may interfere chemically or biochemically with the processes given above, without any limitation.

Preferably, oxygen compounds of chlorine can be used as reactive chlorine compounds, particularly preferably oxyacids of chlorine, salts or derivatives of these oxyacids. Derivatives of reactive chlorine compounds include carbonic acid adducts of oxyacids of chlorine or similar compounds.

Reactive chlorine compounds, in particular oxygen compounds of chlorine, are already used or at least proposed for some pharmaceutical applications. Thus hypochlorites, in particular sodium hypochlorite, are used in dentistry or for treating patients with atopic eczema. Chlorites, including sodium chlorite, are used for treating wounds, wherein its use for the treatment of amyotrophic lateral sclerosis (ALS) is further proposed. Chlorates, in particular sodium chlorate, are used for treating wounds, and perchlorates, in particular potassium perchlorate, are used for treating thyroid disorders. The treatment of SIRS with these compounds has not been proposed to date.

Furthermore, peroxochlorine compounds are known from WO 00/48940 A1 and dichlorine compounds from WO 2005/049483 A2, which are particularly suitable as agents for treating wounds. The treatment of SIRS with these compounds has not been proposed to date.

Surprisingly, it has been established that the peroxochlorine compounds described in WO 00/48940 A1 and the dichlorine compounds known from WO 2005/049483 A2 can be used for the treatment of SIRS.

Document WO 00/48940 A1, filed at the European Patent Office on 18 Feb. 2000 with application number PCT/EP00/01350, in particular the peroxochloric acids, their derivatives and anions and methods of production described therein and the pharmaceutical preparations described therein are mentioned here for disclosure purposes by reference to this publication. Document WO 2005/049483 A2, filed at the European Patent Office on 22 Nov. 2004 with application number PCT/EP2004/013212, in particular the dichloric acids disclosed therein, derivatives, anions and salts thereof as well as methods for their production and the pharmaceutical preparations described therein are mentioned here for disclosure purposes by reference to this publication.

Here the dichloric acids presented in WO 2005/049483 A2 are superior to the peroxochloric acids described in WO 00/48940 A1. This applies in particular to their efficacy and their compatibility as well as their shelf life. Thus dichloric acids are particularly preferred. This also applies to the salts, anions and derivatives of these acids.

Peroxochloric acids or the salts thereof, which are described inter alia in WO 00/48940 A1, and dichloric acids or the salts thereof, which are described inter alia in WO 2005/049483 A2, are superior to hypochlorites, chlorites, chlorates and perchlorates. Thus peroxochloric acids and dichloric acids are preferred, wherein dichloric acids are particularly preferred. This applies in particular to their efficacy and tolerability. This also applies to the salts, anions and derivatives of these acids (peroxochloric acids and dichloric acids).

Preferably, it may be provided that the reactive chlorine compound comprises a peroxochloric acid, a peroxochlorous acid and/or a dichloric acid, preferably a dichloroxo acid, particularly preferably a dichloroperoxo acid or a pharmaceutically tolerable salt of these acids. Furthermore, preferred reactive chlorine compounds include dichloric acids, preferably dichloroxo acids, particularly preferably dichloroperoxo acids, their intermediates such as peroxochloric acid and peroxochlorous acid and their respective derivatives, salts and anions. Here, dichloroxo acids are preferred, wherein dichloroperoxo acids are preferred over other reactive chlorine compounds. Dichloroxo acids are oxyacids of chlorine, which have two chlorine atoms. Dichloroperoxo acids are oxyacids of chlorine, which have two chlorine atoms and at least four, preferably six oxygen atoms.

In a particular embodiment, it may be provided that the reactive chlorine compound comprises a molecular formula selected from HClO, HClO₂, HClO₃, HClO₄ and/or H₂Cl₂O₆ or a pharmaceutically acceptable salt of these acids.

It may further be provided, that the reactive chlorine compound comprises a structure of formula [O−Cl]⁻, [O═ClO]⁻, [(O═)₂ClO]⁻, [O═ClOO]⁻, [O₂ClOO]⁻ and/or [O₂ClOOClO₂]²⁻, preferably [O═ClOO]⁻, [O₂ClOO]⁻ and/or [O₂ClOOOClO₂]²⁻. This structure may be present here as an acid and/or salt, wherein the salt is preferably pharmaceutically acceptable.

Preferably, it may be preferably provided that the reactive chlorine compound comprises a structure of formula [O═ClOO]⁻, [O₂ClOO]⁻, [O₂ClOOClO₂]²⁻ and/or the anion of the reactive chlorine compound has the molecular formula Cl₂O₆²⁻.

Preferably, peroxochloric acids are used with the molecular formula HClO₄, wherein the anion preferably has a structure [O₂ClOO]⁻, which in particular has a peroxo group (O—O). In the case of a preferably used peroxochloric acid, chlorine has the oxidation state of +5, wherein these compounds are described inter alia in WO 00/48940 A1.

Preferably, a reactive chlorine compound, preferably a peroxochloric acid or a salt of this acid, can be obtained by a method in which
(a) chlorine dioxide is reacted with an aqueous or water-containing solution of hydrogen peroxide or another hydroperoxide or peroxide at a pH >=6.5,
(b) the pH is lowered to 3 to 6 by the addition of an acid,
(c) the gaseous free reactive chlorine compound is expelled with a cooled gas and collected in a basic solution with a pH >10.

Further preferred embodiments of this method are presented with reference to the dichloric acids described below and which are used particularly preferably. Here the methods differ in particular regarding step d). Steps a) to c) apply accordingly.

Particularly preferably, dichloric acids are used with the formula H₂Cl₂O₆ and their derivatives, anions, or salts as reactive chlorine compounds. These compounds are also referred to here as dichloroperoxo acids, regardless of the structure of the anion of this acid.

The exact structure of the reactive chlorine compound, in particular of the dichloroperoxo acids is not essential here, wherein the following structures can be assumed. In particular dichloric acids are preferred with the formula H₂Cl₂O₆ and their derivatives, anions, or salts, with the structural formulae of the anions

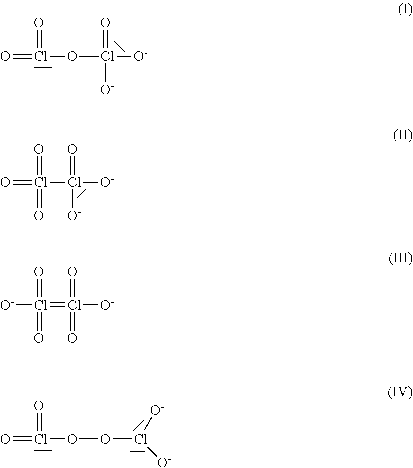

wherein dichloric acids of the anions of the structural formulae I-III are particularly preferred.

Preferred dichloric acids are shown in the following Table 1. Of these dichloric acids, dichloric acids no. 1 to no. 3 represent particularly preferred embodiments of the compounds to be used.

TABLE 1

| | Formal | | |
|---|---|---|---|
| | oxidation states | Structural formula of | Structural formula of |
| No. | of chlorine | the acid | the Di-anion |
| 1 | +5, +5 | 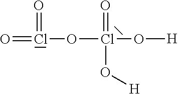 | 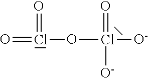 |
| 2 | +6, +4 | 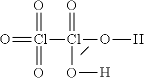 | 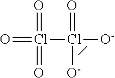 |
| 3 | +5, +5 | 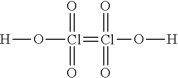 | 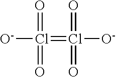 |
| 4 | +5, +3 | 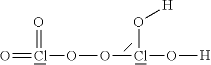 | 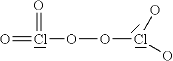 |

In addition to the valances already described +3/+5 (WO 00/48940) and +4/+4 (Bogdanchikov G. A., Kozlov, Y. N. and Berdnikov, V. M. “The Mechanism of the Elementary Act of HO₂-Anion Oxidation by a ClO₂ Radical in Aqueous Solution” Khim.Fiz. 1983 (5), 628-636) the dichloric acids no. 1 to no. 3 used according to the invention with the valences of +6/+4 and +5/+5 for chlorine can also and preferably be used for the production of a pharmaceutical preparation according to the invention.

The reaction of peroxochlorate ions O₂ClOO⁻ with chlorite ions (ClO₂⁻) leads directly to the range of “dimer” Cl₂O₆²⁻ species, which can preferably be used:

O₂ClOO⁻+ClO₂⁻→Cl₂O₆²⁻->-> and isomers

Furthermore, surprisingly the peroxochlorite ion, O═ClOO⁻ and peroxochlorous acid O═ClOOH derived therefrom can be used for the production of pharmaceutical preparations according to the invention.

Particularly around the neutral point, the decomposition of the dichloric species Cl₂O₆²⁻ into chlorate ions ClO₃⁻ and peroxochlorite ions OClOO⁻ is in clear competition with the described intramolecular redox reactions of the dichloric species, leading to compounds 1-4 in the above Table.

When reference is made to anions in the present disclosure, the presence of required counter ions is included (mainly in solution). The term anions is primarily used to express that in solution the dichlorate (Cl₂O₆²⁻) is the more stable form compared to the protonated acid (H₂Cl₂O₆). However, according to the invention, depending on the context, the term “anion” can also be representative of the acid, the term “acid” can also be representative of the “anion”. The counter ions preferably represent pharmaceutically acceptable cations, which are generally known.

The reactive chlorine compounds to be used according to the invention can also be used as a mixture. Thus, dichloric acids and peroxochlorous acid and also the anion present at physiological pH values can therefore be present in solution as a mixture with peroxochlorate and chlorite according to the invention and can be used as such. Such a solution comprising the dichloric acids, peroxochlorous acid, peroxochlorate and chlorite is therefore one of the particularly preferred exemplary embodiments of the present invention.

However, since large amounts of chlorite are detrimental to the use of dichloric acids in the pharmaceutical sector, it is particularly preferred if chlorite is present in the end product of the solutions according to the invention in an excess amount of no more than 20 times, preferably no more than 5 times, and in particular no more than 3 times by weight over other reactive chlorine compounds, in particular dichloric acids, preferably dichloroxo acids, particularly preferably dichloroperoxo acids, relative to the total weight of the solution.

In particular, the preferred dichloric acids to be used and the peroxochlorous acid are present in this solution in amounts of about 0.1-20 wt. %, preferably 3-5 wt. %, based on the weight of ClO₂ used. The qualitative detection is achieved by Raman spectroscopy. Performing this type of spectroscopy is a matter of course for the person skilled in the art in this field. The obtained spectrograms of dichloric acids, preferably dichloroxo acids, particularly preferably dichloroperoxo acids, are significantly different from the compositions which are obtained by the method of WO 00/48940. The quantitative portion can be determined by titration.

It has already been mentioned that dichloric acids, preferably dichloroxo acids, particularly preferably dichloroperoxo acids are preferred over other reactive chlorine compounds, in particular also peroxochloric acid. Accordingly, it is particularly preferred, if peroxochlorate (ClO₄⁻) is present in the end product of the solutions according to the invention in an excess amount of no more than 20 times, preferably no more than 5 times and in particular no more than 3 times by weight over other reactive chlorine compounds, in particular dichloric acids, preferably dichloroxo acids, particularly preferably dichloroperoxo acids, relative to the total weight of the solution. Particularly preferably, the proportion of peroxochlorate (ClO₄⁻) is below 25 wt.-%, preferably below 15 wt-.%, particularly preferably below 5 wt.-%, relative to the weight of the dichloric acids contained.

Further qualitative detection is possible by the reaction with the haem iron. In the presence of preferred dichloric acids, preferably dichloroxo acids, particularly preferably dichloroperoxo acids, the time curve of the change in intensity of the Soret band is markedly different from that of the solutions obtained by the method of WO00/48940.

Particularly preferably, a reactive chlorine compound can be obtained according to a method, in which
(a) chlorine dioxide is reacted with an aqueous or water-containing solution of hydrogen peroxide or another hydroperoxide or peroxide at a pH >=6.5,
(b) the pH is lowered to 3 to 6 by the addition of an acid,
(c) the gaseous free reactive chlorine compound is expelled with a cooled gas and collected in a basic solution with a pH >10, and
(d) the collected reactive chlorine compound is incubated with chlorite at a pH of 6 to 8, preferably approximately 7.

By a method comprising steps (a) to (d) in particular dichloric acids, preferably dichloroxo acids, particularly preferably dichloroperoxo acids are obtained.

The reactive chlorine compounds to be preferably used can be obtained in particular by a method which preferably consists of reacting chlorine dioxide with aqueous or water-containing hydrogen peroxide or another peroxide or hydroperoxide known to a person skilled in the art, e.g. peroxocarbonate or perborate or the urea adduct of hydrogen peroxide at a pH of 6.5 or higher, preferably pH 10-12. It is preferable to keep the pH at a constant value.

It should be noted here that peroxochloric acid and its anions and derivatives which occur as an intermediate, can also be obtained by reacting chlorine dioxide with other oxidizing agents which contain the peroxo grouping.

The reaction can be carried out in an aqueous medium or in a water-containing medium. For example, in addition to water, solvents miscible with water can be used, such as alcohols, e.g. alkanols, such as methanol, ethanol or the like, or mixtures thereof.

Optionally, other chloroxides can also be used. For example, chlorine monoxide, preferably in its dimeric form (Cl₂O₂), can also be reacted with a hydroperoxide (preferably hydrogen peroxide) to obtain the desired product. The reaction works in the same pH range as for chlorine dioxide.

The reaction temperature can be increased, for example up to about 50° C.; in purely aqueous systems the lowest temperature is preferably about 0° C. However, chlorine dioxide should not be used below +10 degrees Celsius, since below this temperature the chlorine dioxide gas liquefies and deflagrations may occur. If there are additional organic solvents and/or high concentrations of the reagents involved, lower temperatures can also be used, i.e. below the freezing point of water. Preferably, this is conducted at room temperature.

The chlorine dioxide required for the reaction is available to the person skilled in the art and can be produced in the usual way. For example, it can be produced by reacting a chlorite with an acid (for example sodium chlorite with sulfuric acid) or by the reduction of chlorate, for example with sulfuric acid.

The chlorine dioxide obtained in this way can be released if necessary after the removal of any traces of chlorine in a known manner (Granstrom, Marvin L.; and Lee, G. Fred, J. Amer. Water Works Assoc. 50, 1453-1466 (1958)).

If the chlorite used for the production of ClO₂ is contaminated with carbonate, this produces ClO₂ contaminated with CO₂, and/or the carbonic acid adducts described in WO00/48940. For the absorption of carbon dioxide, the gas stream containing chlorine dioxide and carbon dioxide should be passed through a wash bottle charged with lye. If contact times are short, the CO₂, but not the ClO₂ will be absorbed by the lye. However, it is better to free the carbonate impurities by fractional crystallization of the sodium chlorite used. Any contamination of the peroxochlorate with carbonate can be easily identified in the Raman spectrum. Instead of the sharp band at 1051 cm-1 a double band is obtained at 1069 cm-1 (broad) and the band at 1051 cm-1 (sharp).

The chlorine dioxide can be carried with an inert gas, such as nitrogen or a noble gas such as argon, but also by air or oxygen to react with the peroxo compound or hydroperoxide, such as the hydrogen peroxide or percarbonate or perborate. For example, it is possible to produce the chlorine dioxide in a first reaction vessel and introduce it with the said gases or a mixture thereof into a second reaction vessel in which the peroxo compound (peroxide or hydroperoxide) is in an aqueous or water-containing solution.

The pH of the reaction mixture is kept equal to or above 6.5 by the addition of a base. It is preferable to keep the pH constant. This can be done either manually or automatically by a “pH-stat” device.

Common inorganic or organic bases, such as alkaline lyes, for example sodium hydroxide or potassium hydroxide, earth alkali hydroxides, ammonia or organic bases such as nitrogen bases, can be used as the bases. The hydroxides of quaternary ammonium salts, in particular alkyl, such as trialkyl or tetraalkyl ammonium hydroxides or zinc hydroxides can also be used.

The content of hydroperoxide in the reaction mixture can be determined for example by potentiometric titration with an acid, such as for example hydrochloric acid.

The solutions obtained according to the method described above can be used as such or also in a modified form. For example, excess hydrogen peroxide can be removed in the usual way, e.g. with a heavy metal compound such as manganese dioxide. Excess amounts of other oxidizing agents can be removed in a similar way.

Excess chlorine dioxide (ClO₂) can be removed with H₂O₂. This should be done as soon as possible, as otherwise by

2ClO₂+2OH⁻->ClO₂⁻+ClO₃⁻+H₂O

the disruptive ClO₃— with pentavalent chlorine (chlorate) would form. However, a product containing chlorate is not preferred over other pharmaceutical preparations, so that the formation of chlorate should be avoided. Particularly preferably, the proportion of chlorate (ClO₃—) is below 25 wt.-%, preferably below 15 wt-.%, particularly preferably below 5 wt.-%, based on the weight of the oxychloric acids obtained.

To improve the shelf life of the reaction product for example storage at an elevated pH value is suitable, for example at a pH of 10 or more. This pH value can be adjusted with a suitable base, as described above for the method of production.

To produce solutions, which contain the preferred dichloric acids and/or peroxochlorous acid and/or the salts of these said acids, it has been made possible surprisingly to expel and capture the free peroxochlorous acid HOOClO, the dichloric acids or the peroxochloric acid when the pH is below 6, e.g. a pH 5 or less from the mixture containing the obtained chlorite ions, with an inert gas, such as a noble gas, e.g. argon or nitrogen or also the gases oxygen or air, particularly preferably the free peroxochlorous acid HOOClO, the dichloric acids and the peroxochloric acid are expelled by an oxygen-rich gas. Surprisingly, it has been shown that the yield can be increased considerably if the gas path is kept very short and the gas flow is cooled.

The mixture produced in step (a) of the aforementioned production method initially contains very high concentrations of chlorite ions (ClO₂—). The content of chlorite can however be significantly reduced by “passing” it in the gas stream into a basic solution. Here, the chlorine acids of all kinds are expelled as volatile compounds in protonated (neutral) form, which are very unstable however. A base is present in the sample, whereby the chlorine acids are depronated and anions are formed. After adjusting the solution to pH 6-8 and after adding defined amounts of chlorite, for example in the form of sodium chlorite, the anions of the more preferred dichloric acids are formed.

Preferred dichloric acids are obtainable by a method in which the reactive chlorine compound obtained according to steps a) to c) described above is incubated with chlorite at a pH of 6 to 8. The incubation time can be selected more or less at random, wherein incubation times that are too short lead to incomplete conversion, and incubation times that are too long lead to possible decomposition of the more preferred dichloric acids. Preferably, the incubation time can be in the range of 1 second to 1 week, particularly preferably in the range of 1 minute to 24 hours and especially preferably 5 minutes to 1 hour. The incubation time can be controlled by adjusting the pH value, wherein an increase of the pH value to above 8, in particular above 9, ends the incubation.

The amount of chlorite can be within a broad range. Large amounts of chlorite in relation to the reactive chlorine compound obtained according to steps a) to c) described above lead to a very complete conversion to the particularly preferred dichloric acids. Small amounts of chlorite in relation to the reactive chlorine compound obtained according to steps a) to c) described above lead to residual amounts of the reactive chlorine compounds obtained according to steps a) to c) described above.

Here, the chlorite can be used in an excess amount of up to 100 times, preferably up to 10 times, with respect to the reactive chlorine oxygen compound obtained according to the aforementioned steps a) to c). In a particularly preferred embodiment, it can be provided that the molar ratio of chlorite to the reactive chlorine oxygen compound obtained according to the aforementioned steps a) to c) is in the range of 10:1 to 1:10, preferably 2:1 to 1:2 and more preferably 1:1 to 1:1.2.

As on the one hand large excess amounts of chlorite should be avoided and on the other hand the dichloric acids obtained by incubation with chlorite are preferable to other reactive chlorine compounds, preferably equimolar amounts of chlorite and of the reactive chlorine oxygen compound obtained according to steps a) to c) described above are used.

For example, it can be collected in a base, such as an alkali metal base, alkaline earth metal or zinc base or nitrogen base, such as ammonia or an organic amine. However, it is also possible to freeze out the gaseous acids in a cold trap (e.g. at −100 to −190° C.).

All metal cations and organic cations, such as those of nitrogen bases, in particular quaternary ammonium salts, can be used as counter ions. For pharmaceutical applications, alkaline earth metals or alkali metals are preferred in particular, preferably Na⁺ or K⁺, or Zn²⁺.

It is expedient and preferred to store the acids according to the invention and their derivatives, salts and/or anions with the exclusion of light and to produce aqueous solutions from them with high pH values, e.g. with pH values of 10, 11 or 12 and above, in particular in the range of pH 10 to pH 13, in order to achieve long-term storage capabilities. From such solutions, the free acid can be recovered as required, as described above, and if necessary converted into solutions with a desired pH or into salts.

Preferably, the pharmaceutical preparation shows a signal in a mass spectrum at 189.0 m/z. This signal is preferably based on the reactive chlorine compound used. Accordingly, reactive chlorine compounds are preferred, which show a signal in the mass spectrum at 189.0 m/z.

Furthermore, the mass spectrum of the pharmaceutical preparation may have a signal at 99 m/z, wherein the signal is preferably based on the reactive chlorine used.

In a preferred embodiment of the pharmaceutical preparation or the reactive chlorine compound, it may be provided that the signal at 189.0 m/z of the mass spectrum of the pharmaceutical preparation is higher than at 99 m/z.

Furthermore, the mass spectrum of the pharmaceutical preparation may have a signal at 83.2 m/z, wherein the signal is preferably based on the reactive chlorine compound used.

In a preferred embodiment of the pharmaceutical preparation or the reactive chlorine compound, it may be provided that the signal at 83.2 m/z of the mass spectrum of the pharmaceutical preparation is higher than at 99 m/z.

The mass spectrum can be obtained by conventional methods, wherein it is preferably performed according to the method given in the examples.

Furthermore, a preferred pharmaceutical preparation for use according to the present invention may have a reactive chlorine compound which, in an ion chromatogram, shows a peak at a retention time of 15 min. The ion chromatography can be performed using conventional methods, wherein this is preferably performed according to the method given in the examples.

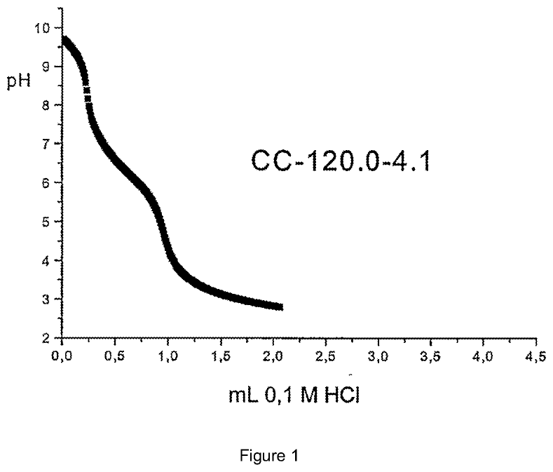

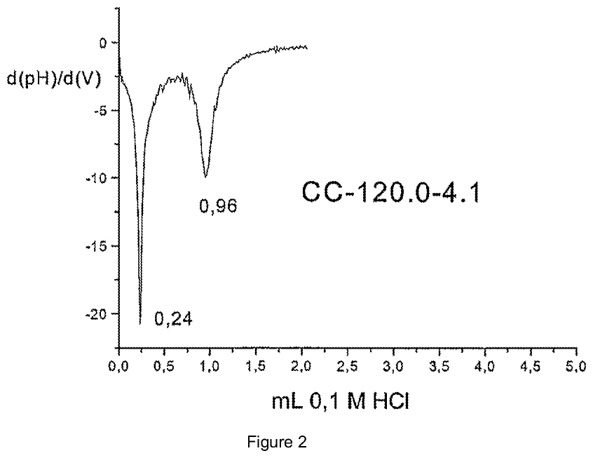

The particularly preferred dichloric acids preferably have two transitions in a titration curve, as shown in FIGS. 1 and 2. This titration curve shows two transitions, which can be assumed to be caused by the pKa values of the particularly preferred dichloric acids. Accordingly, the particularly preferred dichloric acids preferably have two pKa values, wherein one pKa value is in the region of 8.5 and one pKa value is in the region of 5. These values are determined potentiometrically at 25° C. with a 0.1 M hydrochloric salt.

For comparison, pKa values of further reactive chlorine compounds are listed below:

| | | | | Acid |
|---|---|---|---|---|
| | Name of the | | | dissociation |
| Oxidation | acid and its | Formula | | constant as |
| number | salts | of the acid | Anion | pKa at 298 K⁾ |
| +I | hypochlorous | HOCl | hypochlorite | 7.25 |
| | acid | | OCl⁻ | |
| | hypochlorites | | | |
| +III | chlorous acid | HOClO | chlorite | 2 |
| | chlorites | | ClO₂⁻ | |
| +V | chloric acid | HOClO₂ | chlorate | 0 |
| | chlorates | | ClO₃⁻ | |
| +VII | perchloric acid | HOClO₃ | perchlorate | −9 |
| | perchlorates | | ClO₄⁻ | |
| +V | peroxochloric | HOOClO₂ | peroxochlorate | approx. 6.2 |
| | acid | | —OOClO₂ | |

The dichloric acids, or the peroxochlorous acid, their respective derivatives, or anions and salts, which are preferred according to the invention, can be used as such and in particular in aqueous or water-containing solution as a pharmaceutical preparation for use in the treatment of systemic inflammatory response syndrome (SIRS).

The preparations can contain the active ingredient alone or preferably together with one or more pharmaceutically applicable carriers.

The pharmaceutical preparation can preferably comprise a pharmaceutically acceptable carrier. The pharmaceutically acceptable carrier is preferably adapted to the form of administration. Preferably, the pharmaceutical carrier comprises water, wherein the water content is preferably at least 90 wt.-%, based on the weight of the pharmaceutical carrier. In a preferred embodiment, it may be provided that the pharmaceutical preparation is an aqueous solution. The aqueous solution here comprises the reactive chlorine compound described above.

Preferably, it may be provided that the reactive chlorine compound is present in the pharmaceutical preparation in a molar concentration of at least 0.01 mmol/l, preferably at least 0.1 mmol/l, particularly preferably at least 0.3 mmol/l and especially preferably at least 0.5 mmol/l, wherein preferably water is used as the pharmaceutically acceptable carrier and the amount of reactive chlorine compound is determined by titration. The concentration of reactive chlorine compound can be determined by any suitable method, wherein preferably titration is performed with HCl. The concentration of HCl in the titration solution is preferably in the range of 0.01 to 1 mold, particularly preferably about 0.1 mold. Here, the concentration of reactive chlorine compound is determined by the pH value of the composition. Further details on this can be taken from the examples.

Preferably, it can be provided that the reactive chlorine compound is present in the pharmaceutical preparation in a molar concentration in the range of 0.01 mmol/l to 100 mmol/1, preferably 0.1 mmol/l to 50 mmol/1, particularly preferably 0.3 to 20 mmol/1 and especially preferably from 0.5 mmol/l to 10 mmol/1, wherein water is preferably used as the pharmaceutically acceptable carrier and the proportion of reactive chlorine compound is determined by titration. The molar concentration can preferably be determined by titration with HCl, as explained in more detail above.

Preferably, it may be provided that the reactive chlorine compound is present in the pharmaceutical preparation in a concentration by weight of at least 2 mg/l, preferably at least 20 mg/l, particularly preferably at least 60 mg/l and especially preferably at least 100 mg/l, wherein preferably water is used as the pharmaceutically acceptable carrier and the amount of reactive chlorine compound is determined by titration. The concentration of reactive chlorine compound can be determined by any suitable method, wherein preferably titration is performed with HCl, as described above. The molar mass of the reactive chlorine compound is used for the conversion, wherein in a preferred embodiment, in particular in the case of dichloric acids with a molecular formula Cl₂O₆²⁻ this can be taken to be about 167 g/mol. Preferably, the anion can be used as the basis.

Preferably, it can be provided that the reactive chlorine compound is present in the pharmaceutical preparation in a molar concentration in the range of 2 mg/l to 20000 mg/l, preferably 20 mg/l to 10000 mg/l, particularly preferably 600 mg/l to 4000 mg/l and especially preferably 100 mg/l to 2000 mg/l, wherein water is preferably used as a pharmaceutically acceptable carrier and the amount of reactive chlorine compound is determined by titration. The concentration of reactive chlorine compounds based on the weight can preferably be obtained by titration with HCl and a subsequent conversion, taking into account the molar mass, wherein particularly preferably the molar mass of the anion is used.

In addition to a reactive chlorine compound a pharmaceutical preparation may comprise at least one further active ingredient, which is different from a reactive chlorine compound, as this is the subject-matter of the present application. A further subject-matter of the present invention is a pharmaceutical preparation having at least one reactive chlorine compound, as described above and in the following, and at least one pharmaceutically active substance, which differs from the reactive chlorine compound given above and in the following.

Preferred further active ingredients other than a reactive chlorine compound include, but are not limited to antibiotics, antipyretics, drugs for the treatment of disseminated intravascular coagulation (DIC), antibodies, cytokines, chemokines, antimicrobial peptides, sphingomyelinase inhibitors, statins, alpha-2-macroglobulin, thrombin-derived C-terminal peptide, sphingosine-1-phosphate, curcumin, ascorbic acid, resveratrol, melatonin, glycyrrhizin and erythropoietin. These active ingredients can be used individually or as a mixture of two, three, four or more ingredients.

Preferred antibiotics include inter alia β-lactam antibiotics, such as penicillins, in particular benzylpenicillin, phenoxymethylpenicillin, propicillin, azidocillin, flucloxacillin, dicloxacillin, cloxacillin, oxacillin, methicillin, amino penicillins, such as amoxicillin, ampicillin and bacampicillin, acylamino penicillins, such as mezlocillin and piperacillin, pivmecillinam; cephalosporine, basiscephalosporine, cefuroxim, cefamandol, cefoxitin, cefotiam, cefotaxim, cefovecin, ceftazidim, cefepim, cefodizim, ceftriaxon; oralcephalosporine, cefaclor, cefadroxil, cefalexin, loracarbef, cefixim, cefuroximaxetil, cefetametpivoxil, ceftibuten, cefpodoximproxetil; β-lactamase inhibitors, in particular sulbactam, clavulan acid in combination with amoxicillin, tazobactam in combination with piperacillin, carbapeneme, imipenem in combination with cilastatin, meropenem, doripenem, ertapenem, monobactame, such as aztreonam.

Furthermore, preferred antibiotics include glycopeptides, such as vancomycin, dalbavancin and teicoplanin.

Preferred antipyretics include inter alia non-steroidal antirheumatic drugs, such as ibuprofen, naproxen, nimesulide and ketoprofen (arylpropionic acid derivatives), acetylsalicylic acid; paracetamol (aminophenol derivatives); pyrazolone derivates, such as phenazone, propyphenazone, metamizole; nabumetone (arylacetic acid derivatives) and quinine.

Preferred drugs for the treatment of disseminated intravascular coagulation (DIC) include antithrombin, protein C and APC (activated protein C), thrombomodulin (TM), heparin, tissue factor pathway inhibitor (TFPI).

Preferred antibodies include inter alia immunoglobulins, cytokine inhibitors, in particular antibodies against IL-1, IL-17A or IL-18; antibodies or other molecules, which block the binding of PD-L1 to its receptor PD-1 (programmed cell death-1); and antibodies and other blocking molecules directed against surface proteins on thrombocytes (abciximab, tirofiban, and eptifibatide) and immune cells (CD39).

Preferred cytokines include inter alia interleukin 7 (IL-7), IL-15 and GM-CSF (granulocyte-macrophage colony stimulating factor).

Preferred chemokines include inter alia CXCL10.

Preferred antimicrobial peptides include inter alia thymosin alpha 1.

Preferred sphingomyelinase inhibitors include inter alia amitriptyline.

Preferred statins include inter alia atorvastatin and simvastatin.

The dosage of the reactive chlorine compound to be used according to the invention may be selected according to the symptoms and the condition of the patient. In one embodiment, the pharmaceutical preparation can be provided in a form which is administered in a single dose. Preferably, the pharmaceutical preparation is provided in the form of a preparation that can be administered multiple times. For example, it may be provided that the pharmaceutical preparation can be administered at least twice, preferably at least three times, particularly preferably at least four times and especially preferably at least five times.

Preferably, the pharmaceutical preparation is provided such that there is a period of 1 hour to 5 days, preferably 2 hours to 2 days and especially preferably 3 hours and 1 day between the administrations.

The dosage of the reactive chlorine compound to be used according to the invention depends on the symptoms to be treated, as a well as on the species, their age, weight and individual condition, individual pharmacokinetic conditions as well as the mode of application. Preferably, the dosage for parenteral application (for example by infusion or injection) (preferably in humans) is in the range of 0.01 to 100 pmol/kg, in particular between 0.1 to 100 pmol, i.e. for example in a human with a body weight of 70 kg a 1 mg to 1 g/day, in particular 8.5 mg to 850 mg/day, in a single dose or divided into several doses.

The dosage of further active ingredients, which are different from a reactive chlorine compound, can be chosen according to the symptoms to be treated, as well as species, their age, weight and individual condition, individual pharmacokinetic conditions as well as a the mode of application, wherein these can be selected in accordance with the usual dose/doses for the other active ingredients.

The invention also relates to a pharmaceutical composition for the prophylactic and in particular therapeutic treatment of disease states described therein, preferably for prophylactic or therapeutic treatment, which are associated with systemic inflammatory response syndrome (SIRS), preferably of a warm-blooded animal suffering from such a disease, containing one or more reactive chlorine compounds, preferably peroxochloric acid, peroxochlorous acid and/or dichloric acids, particularly preferably dichloroxo acids and/or peroxochlorous acid, more preferably dichloroperoxo acids, or their respective derivatives or salts in an amount effective prophylactically or in particular therapeutically to combat said disease and one or more pharmaceutically applicable carriers.

The invention also relates to a method for treating disease conditions, preferably for prophylactic and/or therapeutic treatment—in particular in a warm-blooded animal, in particular a human—which are associated with systemic inflammatory response syndrome (SIRS), comprising the administration of reactive chlorine compounds, preferably peroxochloric acid, peroxochlorous acid and/or dichloric acids, particularly preferably dichloroxo acids and/or peroxochlorous acid, more preferably dichloroperoxo acids, or their respective derivatives, anions or salts in an amount effective against said diseases to a warm-blooded animal, e.g. humans, in need of such treatment.

The invention also relates to the use of reactive chlorine compounds, preferably peroxochloric acid, peroxochlorous acid and/or dichloric acids, preferably dichloroxo acids and/or peroxochlorous acid, particularly preferably dichloroperoxo acids, and their derivatives, anions or salts for use in a method for treating the human or animal body to combat SIRS.

The invention relates in particular to the use of reactive chlorine compounds, preferably peroxochloric acid, peroxochlorous acid and/or dichloric acids, preferably dichloroxo acids and/or peroxochlorous acid, particularly preferably dichloroperoxo acids, their derivatives, anions or salts for the production of a drug for the treatment of the human or animal body, preferably for the prophylactic and/or therapeutic treatment—in particular in a warm-blooded animal, in particular a human—of systemic inflammatory response syndrome (SIRS).

Dosage forms include e.g. ampoules, vials, syringes or sachets. Further forms of application, in particular for solutions of reactive chlorine compounds, preferably dichloric acids, preferably dichloroxo acids, particularly preferably dichloroperoxo acids or peroxochlorous acid, their ions, derivatives or salts, include e.g. drops, sprays, and the like. The dosage forms, e.g. ampoules, vials, syringes or sachets, contain preferably about 0.005 g to 10.0 g, in particular 8.5 mg to 850 mg, of a salt of reactive chlorine compounds, preferably peroxochloric acid, peroxochlorous acid and/or dichloric acids, particularly preferably dichloroxo acids and/or peroxochlorous acid, more preferably dichloroperoxo acids, their anions, derivatives with usual carriers.

In a preferred embodiment, it may be provided that the pharmaceutical preparation is provided in the form of a preparation which can be administered intravenously.

It may be further provided that the pharmaceutical preparation is provided in the form of depository, which can be inserted into a body.

Furthermore, it may be provided that the pharmaceutical preparation is provided in the form of an inf usable preparation.

Furthermore, it may be provided that the pharmaceutical preparation is provided in a sachet, which comprises at least two compartments for storing at least two liquids, which can be opened by a mechanical action, so that the liquids can be mixed after opening the compartments, wherein one of the compartments contains a liquid a reactive chlorine compound of the present invention and one of the compartments contains a liquid which is adjusted for setting the pH value to a physiological pH value. This method of delivery is particularly useful for reactive chlorine compounds, whose pH has to be adjusted to a physiologically acceptable value. This applies, inter alia to the oxoacids of chlorine described above which have a good shelf life at a high pH. If another active ingredient, which is different from a reactive chlorine compound, is used with it, this can be stored in one of the two compartments, depending on its stability. Preferably however, this is provided in a third compartment, the sachet being designed such that firstly the pH of the reactive chlorine compound is adjusted to a physiologically acceptable value, after which the obtained mixture is mixed with the contents of the third compartment. Sachets that are suitable for this purpose are known from the prior art and can be acquired commercially in many forms. Preferred sachets are described inter alia in WO 2008/155112 A1 and WO 95/26177 A1. Document WO 2008/155112 A1, filed at the European Patent Office on 19 Jun. 2008 with the application number PCT/EP08/004911, particularly the sachets described therein as well as methods for the production thereof, are referred to here for disclosure purposes as reference. Document WO 95/26177 A1, filed at the European Patent Office filed on 28 Mar. 1995 with the application number PCT/EP95/01152, particularly the sachets described therein and methods for the production thereof, are referred to here for disclosure purposes as reference.

The pharmaceutical preparations of the present invention are produced in a known manner, e.g. by means of conventional mixing, dissolution, or lyophilization methods.

In a preferred embodiment, a 0.005 to 1 M solution of one or more reactive chlorine compounds, preferably peroxochloric acid, peroxochlorous acid and/or dichloric acids, particularly preferably dichloroxo acids and/or peroxochlorous acid, more preferably dichloroperoxo acids and/or a salt of these acids or their derivatives can be dissolved in double-distilled water at a pH equal to or >10, preferably 10 to 13, in particular 12.5. Directly before administration, this solution is diluted to isotonicity to concentrations of about 1-10 mM with common salt, sodium or potassium bicarbonate and double-distilled water and approximated to the physiological pH. This solution is suitable for parenteral, preferably intravenous use.

For a preferred formulation of a drug for parenteral use, the reactive chlorine compounds, preferably peroxochloric acid, peroxochlorous acid and/or dichloric acids, particularly preferably dichloroxo acids and/or peroxochlorous acid, very particularly preferably dichloroperoxo acids, or their derivatives as salts are dissolved in double-distilled water with concentrations in the lower millimolar or upper micromolar range, preferably in a concentration range of 0.5-10 mM with a pH equal to or >10, in particular 10 to 13, preferably e.g. pH 11.5 and adjusted to isotonicity with glycerine, common salt or another suitable compatible, preferably physiological agent. Before use, a physiological pH value is adjusted with a physiologically acceptable acid, preferably HCl. Further additives are possible. In particular, when filling the drug into plastic containers, additives are suitable which can neutralize traces of transition metals, since transition metals are dissolved into the walls during storage and can catalyze a decomposition of the active ingredient. Examples of such additives are oligo- or polyalcohols, such as ethylene glycol, desferrioxamine or EDTA (e.g. as disodium EDTA). The solution obtained in this way can be administered directly, preferably parenterally, particularly preferably intravenously.

The anions of the preferably used dichloric acids or peroxochlorous acid are stable, the acids themselves decompose relatively quickly. The stabilization of the active pharmaceutical ingredient can therefore be achieved via the pH value. The active ingredient solution can be lowered to an almost physiological value by buffer dilution immediately before use to improve tolerability.

Since the dichloric acids or the peroxochlorous acid to be used are defined compounds, there should also be no difficulties regarding the approval of the drug.

The pharmaceutical preparation is used in particular for the treatment of systemic inflammatory response syndrome (SIRS). SIRS can be caused by an infection, for example by bacteria, fungi, parasites, viruses and other pathogens. Furthermore, SIRS can also have causes that are not due to an infection. The pharmaceutical preparation of the present invention can be used for the treatment of any form of SIRS, in particular systemic inflammatory response syndrome (SIRS) associated with a bacterial infection. In this case, it is often referred to as sepsis.

The pharmaceutical preparation of the present invention is characterized by excellent tolerability, so that it can also be used in cases of mild or moderately severe SIRS or sepsis.

In a special application it may be provided that systemic inflammatory response syndrome (SIRS) meets at least two of the following criteria:
a) body temperature is either elevated (>38° C.) or depressed (<36° C.);
a) tachycardia (heart rate >90/min)
c) tachypnoea (respiratory rate >20/min) or arterial carbon dioxide partial pressure PaCO₂<33 mm Hg (Torr); equivalent to <4.3 kPa;
d) leukocytosis (leukocytes >12,000/μL) or leukopenia (<4,000/μL).

Furthermore, the pharmaceutical preparation of the present invention is characterized by excellent efficacy, so that it can also be used for severe or very severe forms of SIRS or sepsis. Severe or very severe forms of SIRS are often referred to as septic shock.

Preferably, it can be provided that systemic inflammatory response syndrome (SIRS) is characterized by a SOFA score (SOFA: Sequential Organ Failure Assessment) of at least 2, preferably at least 3. The SOFA score is used to assess the function of six organs or body functions. This assessment is used to classify the body functions of patients receiving intensive care. A value of 0 means a normal ability of the respective body function, a value of 4 means a massive restriction.

Respiratory activity (lungs)

| PaO₂/FiO₂ (mmHg) | Points |
|---|---|
| <400 | 1 |
| <300 | 2 |
| <200 and respiration | 3 |
| <100 and respiration | 4 |

Central nervous system

| Glasgow Coma Score | Points |
|---|---|
| 13-14 | 1 |
| 10-12 | 2 |
| 6-9 | 3 |
| <6 | 4 |

Cardiovascular

| Mean arterial blood pressure (MAP) or | |
|---|---|
| use of vasopressors | Points |
| MAP < 70 mmHg | 1 |
| Dopamine ≤ 5 μg/kg/min or dobutamine | 2 |
| (dosage not important) | |
| Dopamine > 5 μg/kg/min or adrenaline ≤ | 3 |
| 0.1 μg/kg/min or noradrenaline ≤ 0.1 | |
| μg/kg/min | |
| Dopamine > 15 μg/kg/min or adrenaline > | 4 |
| 0.1 μg/kg/min or noradrenaline > 0.1 | |
| μg/kg/min | |

Liver function

| | |
|---|---|
| Bilirubin mg/dl | Points |

| | | |
|---|---|---|
| 1.2-1.9 | mg/dl | 1 |
| 2.0-5.9 | mg/dl | 2 |
| 6.0-11.9 | mg/dl | 3 |
| >12.0 | mg/dl | 4 |

Clotting

| Thrombocytes × 10³/μl | Points |
|---|---|
| <150 | 1 |
| <100 | 2 |
| <50 | 3 |
| <20 | 4 |

Kidney function

| | |
|---|---|
| Creatinine mg/dl | Points |

| | | |
|---|---|---|
| 1.2-1.9 | mg/dl | 1 |
| 2.0-3.4 | mg/dl | 2 |
| 3.5-4.9 | mg/dl | 3 |
| >5 | mg/dl | 4 |

The SOFA score can preferably be determined over a longer time period and at fixed intervals, for example one value every 4, 6, 12 or 24 hours over the treatment period. Here the sums of all body functions or the value of a single body function can be used. Furthermore, the development of the SOFA score can be observed for a single body function or for all body functions.

Preferably, it can be provided that systemic inflammatory response syndrome (SIRS) is characterized by a SOFA score (SOFA: Sequential Organ Failure Assessment) of at least 2, preferably at least 3 for at least one organ or one body function, preferably the lungs or the cardiovascular system. Furthermore, it may be provided that systemic inflammatory response syndrome (SIRS) is characterized by a SOFA-score (SOFA: Sequential Organ Failure Assessment) of at least 4, preferably at least 6 for the sum of the six body functions mentioned above.

The pharmaceutical preparation is characterized by very good tolerability and few side effects. Here, the reactive chlorine compound can be combined with other treatment methods and/or other active ingredients to improve the patient's chances of survival. Preferably, the treatment of SIRS in a human subject can comprise the simultaneous or sequential administration of a haemodialysis therapy to the subject.

A further subject-matter of the present invention is the use of a reactive chlorine compound for the production of a drug for the prophylactic and/or therapeutic treatment of SIRS.

A further subject-matter of the present invention is a combination preparation comprising separate packages of at least one pharmaceutical preparation for use for the treatment of SIRS according to the present invention and at least one drug, which differs from the previously given reactive chlorine compound, as this is the subject-matter of the present application.

Preferred, further drugs, other than a reactive chlorine compound, include inter alia antibiotics, antipyretics, drugs for treating disseminated intravascular coagulation (DIC), antibodies, cytokines, chemokines, antimicrobial peptides, sphingomyelinase-inhibitors, statins, alpha-2-macroglobulin, thrombin-derived C-terminal peptide, sphingosine phosphate, curcumin, ascorbic acid, resveratrol, melatonin, glycyrrhizin and erythropoietin, as previously mentioned above.

The following examples explain the invention in more detail, but are not in any way restrictive.

FIG. 1 shows the titration of the anions of peroxo acids (dichloric acid, peroxochlorous acid) present in the solution to determine the concentration of acid anions.

FIG. 2 shows the derivation of the titration curve of FIG. 1, which is used for the exact determination of the concentration.

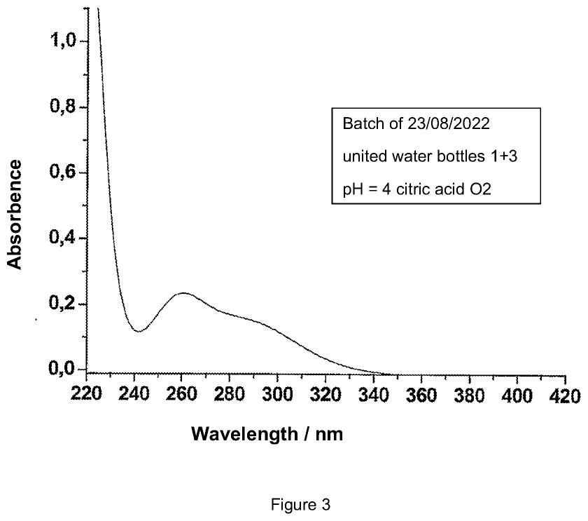

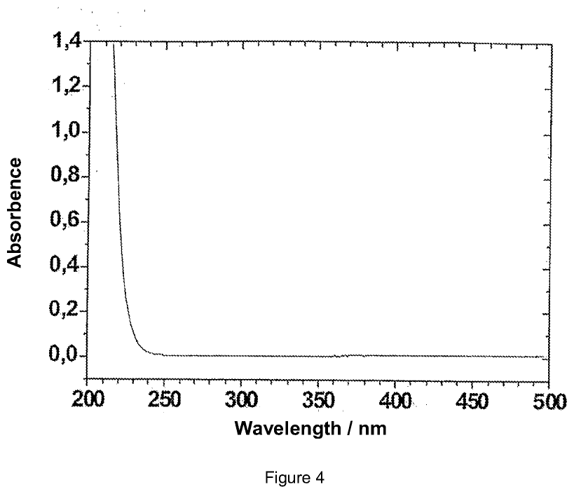

FIGS. 3 and 4 show examples of UV spectra. The UV absorption measurements allow the concentration of existing chlorite to be determined and indicate any dissolved free chlorine dioxide present.

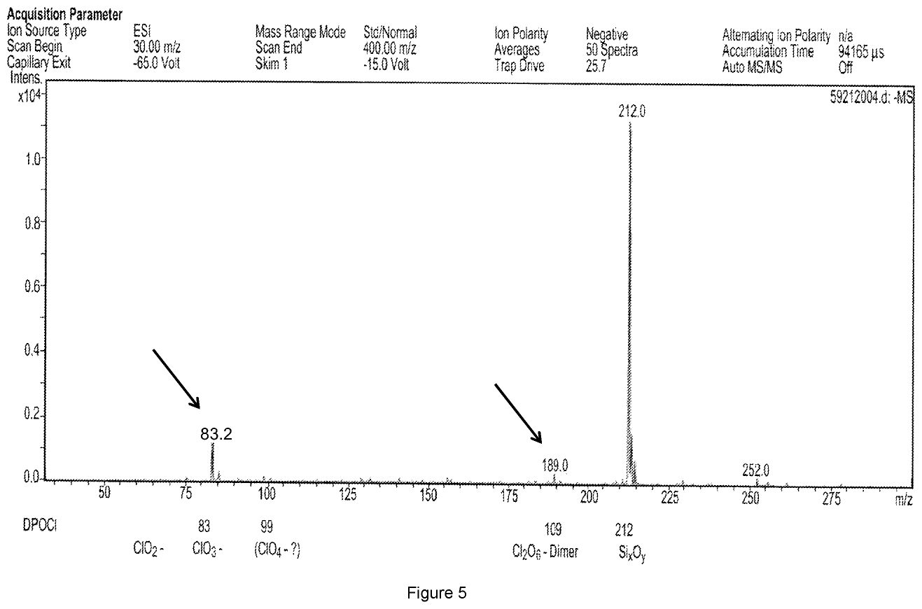

FIG. 5 shows a mass spectrum of the product solution, wherein the peroxochlorite (mass 83.2) and the anion of dichloric acid (mass 189) were detected.

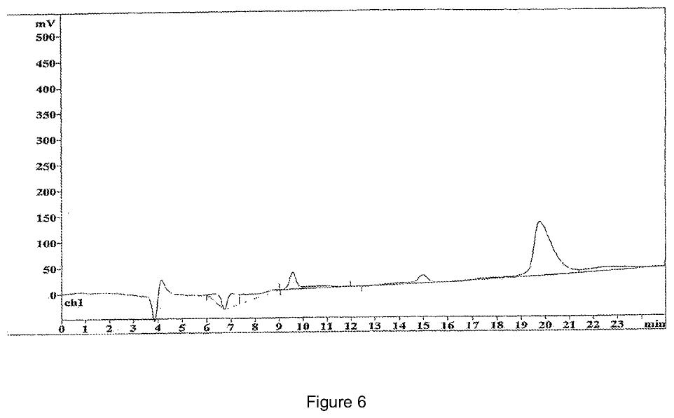

FIG. 6 shows the result of ion chromatography. The retention times of comparative substances are given in example 4, part 5. The dichloric acid is detected at 19.77 min, and no chlorate (ClO₃⁻) is detectable, which rules out chlorate as the cause of the peak in the mass spectrum at 82.3 in FIG. 5.

### EXAMPLE 1: PREPARATION OF DICHLORIC ACIDS

Drops of sulfuric acid (96%) are added carefully while stirring to a solution of 100 g anhydrous sodium chlorite in 200 mL water. With a strong gas flow (Ar, N₂ or O₂ or CO₂-free air) the chlorine dioxide produced is expelled. The gas flow has to be so strong that the ClO₂ content does not rise above 5 percent (risk of explosion). The ClO₂-containing gas flow, is introduced into a solution of 15 mL 30% hydrogen peroxide in 35 mL water, which has previously been brought to pH 12 by the addition of 4M sodium hydroxide solution, via three washing bottles connected in series, each of which is filled with 30 mL of a 2 M NaClO₂ solution with pH 11, in order to capture elemental chlorine. Instead of hydrogen peroxide also a solution of sodium perborate or sodium percarbonate or another peroxo compound can be used such as the H₂O₂ adduct of urea. During the introduction of gas the pH is checked by a glass electrode. By adding 4M NaOH, the pH is kept at 12 for the course of the reaction. The supplied hydroperoxide or supplied peroxo compound is used up when the introduction of gas leads to a permanent yellow coloration. The yellow solution is then decolored again with a drop of the solution of the oxidizing agent (e.g. H₂O₂).

The solution containing reactive chlorine is dropped while stirring to a solution of 500 g citric acid in 3 liters of water, which has been previously adjusted to pH 4.5 with 2 M sodium hydroxide solution. During the addition, the reactive chlorine compound formed is expelled by a powerful gas flow (N₂ or O₂). The gas flow should preferably be cooled. The hose connections should be as short as possible. The gas is collected for example in three wash bottles connected in series, each charged with 50 mL 0.1 M NaOH.

The contents of the wash bottles are combined and kept at pH >10.

For forming the dichloric acids which are preferably used according to the invention the pH is adjusted to 7 for example with hydrochloric acid and a 10-fold molar excess amount of sodium chlorite is added.

In a further embodiment, the pH is adjusted to 7 for example with hydrochloric acid and an equimolar amount of sodium chlorite is added for the formation of the dichloric acids which are preferably used according to the invention.

For storage, it is then preferred in each case if the pH is adjusted to approximately 10 to 13.

The total content of reactive chlorine anions is determined by potentiometric titration with 0.1 M HCl in a manner well known to the person skilled in the art. Here, different compounds can be determined on the basis of the pKa values of the different anions obtained over the titration curve.

The dichloric acids formed are present in solution in a mixture with a defined amount of chlorite and other reactive chlorine compounds.

The presence of the dichloric acids is detected by Raman spectroscopy.

### EXAMPLE 2: ANALYTICAL DETERMINATIONS OF THE SOLUTION OBTAINED FROM EXAMPLE 1

### 1) pH Measurement:

The pH is determined by the single rod glass electrode. The product content and the state of equilibrium are dependent on the pH value.

2) Titration with 0.1 M HCl:

The titration is used for example for quantitatively determining the dichloric acid content or also the content of peroxochlorous acid or peroxochlorate.

1 mL of the product solution is titrated potentiometrically with 0.1 M hydrochloric acid. Titration curves (pH vs. mL 0.1 M HCl) are recorded. From the acid consumption between pH 8.5 and 4.5 determined in the derivation of the titration curve, the content of anions of the corresponding acids in total is determined.

In a typical result 1 mL product solution gives a consumption of 0.72 mL 0.1 M HCl and thus a concentration of 0.072 M.

FIG. 1 shows a recorded titration curve

FIG. 2 shows the derivation of the titration curve and determination of the concentration.

### 3) UV-Vis Absorption Spectrum:

The measurement of the UV spectrum is used to quantify the content of chlorite in the product solution. For comparison, spectra of a chlorite-containing and a chlorite-free product solution are shown in FIGS. 3 and 4 respectively. The chlorite signal is at 260 nm; chlorine dioxide from the process shows a signal at 360 nm.

In 1 cm quartz cuvettes the absorbance values are determined at 260 nm and 500 nm. From the difference A260-A500 and by means of the extinction coefficient for chlorite of c260 nm=140 M-1 cm-1 at 260 nm, the content of ClO₂⁻ ions can be determined.

Absorption at 360 nm indicates free chlorine dioxide (c360 nm=1260 M⁻¹ cm⁻¹).

### 4) Mass Spectroscopy

The ESI mass spectrometry was performed with a Bruker Esquire-LC spectrometer in standard MS mode. The sample was an aqueous product solution diluted with methanol before measurement. The scan range used was between 30 m/z and 400 m/z, with capillary exit −65 volts and skim −15 volts; the spectrum represents an average of 50 measurements.

The right arrow in FIG. 5 indicates the signal of the dichloric acid (molecular formula: Cl₂O₆²), the left arrow shows the signal of the peroxochlorite species (molecular formula: ClO₃⁻).

### 5) Ion Chromatography

All analyses were carried out with a modular ion chromatography system of the company Metrohm.

### Pump: Metrohm IC 709 Pump

### Detector: Metrohm 732 IC Detector

### Suppressor: Metrohm 753 Suppressor Module

### Column: Metrosep A 250

Flow rate: 1 ml/min
Injection volume: 20 μL

### Eluent: 1 mM NaOH

Fresh solutions of reference substances of known concentration were prepared immediately before each measurement and then measured using the method described above with the specified eluent.

### Retention Times of the Reference Substances:

| Substance | Retention time [min] |
|---|---|
| NaCl | 13.21 |
| NaClO₂ | 12.30 |
| NaClO₃ | 16.26 |
| NaClO₄ | 4.36 |
| NaOH | 17.32 |
| Na₂CO₃ | 21.98 |
| Na₂Cl₂O₆ | 19.77 |

FIG. 6: in ion chromatography, dichloric acid yields a typical peak at a retention time of 19.77 min. None of the known reference substances could be detected. The ion chromatography confirms the observation from mass spectroscopy. A chlorate-typical peak (NaClO₃, retention time 16.26 min) is not detectable in the solution prepared according to Example 1. Therefore, the peak of the molecular formula ClO₃-in the mass spectroscopy (FIG. 5, mass 83.2) can only be the dichloroperoxo acid or its ion, which is particularly preferable to use.

### EXAMPLE 3 AND COMPARATIVE EXAMPLE 1

Experiments on mice to demonstrate the efficacy of reactive chlorine compounds to be used according to the invention. The solution of reactive chlorine compounds prepared in Example 1 and examined in more detail in Example 2 will be abbreviated to DPOCL in the following.

It is known from toxicological experiments that 45 mg DPOCL/kg does not cause any clinical symptoms. In order to test whether DPOCL protects from SIRS, the following experiments were carried out. These experiments are based on a CLP model, in which cecal ligation and puncture (CLP) is performed. This animal model is widely recognized and described in more detail by Wichterman et al. (Wichterman K A, Baue A E, Chaudry I H. Sepsis and septic shock—a review of laboratory models and a propsal. Journal of Surgical Research, 114:740-5, 1979). Furthermore, this method has been described above.

NMRI mice receive brief anesthesia with ketamine and xyiazine (ketamine 120 mg/kg KGW; xyiazine 16 mg/kg KGW). After the anesthetic has taken effect they are shaved on the abdominal side and disinfected. The abdominal cavity is opened with a 1 cm incision. The appendix is exposed. Distally, a portion is ligated and perforated with a 0.9 mm cannula. On applying light pressure a small amount of feces comes out. The appendix is pushed back into the abdomen. The peritoneum and musculature are closed with absorbable sutures and the skin is stapled with Michel wound staples. The procedure takes about 8 minutes. Immediately after the procedure and for the next 2 days, the animals are given the analgesic buprenorphine (0.1 mg/kg, s,c.) 2 times daily. Ketamine/xylazine keep the animal under anesthesia for about 45 minutes.

The symptoms of sepsis are assessed according to the following scheme, which is commonly used, wherein the symptoms of sepsis result from a change in behavior (separation, no grooming, passivity) and a drop in temperature:

| | | | Expected | Time | |
|---|---|---|---|---|---|
| Clinical | | | time period in | intervals for | Special |
| model | Symptoms • | Score | experiment¬ | monitoring | measures |
| CLP | no particular | 0 | Day 0 | 2 times | Buprenorphine |
| | findings | | | daily | |
| | Slowed gait, | 1 | from day 1 | 2 times | Buprenorphine; |
| | weight difference | | | daily | Food and water |
| | up to 10% | | | | on the cage floor |
| | | | | | additionally |
| | | | | | accessible |
| | Animal isolates | 2 | from day 1 | 2 times | Buprenorphine; |
| | itself, temperature | | or 2 | daily | Food and water |
| | still above 32° C. | | | | on the cage floor |
| | (temperature | | | | additionally |
| | measured without | | | | accessible |
| | contact with an | | | | |
| | infrared | | | | |
| | thermometer on | | | | |
| | the ventral side), | | | | |
| | weight difference, | | | | |
| | over 10% | | | | |
| | Animal isolates | 3 | from day 1 | 3 times | Buprenorphine |
| | itself and | | or 2 | daily | food and water |
| | temperature | | | | on the |
| | below 32° C.; | | | | |
| | reluctance to move; | | | | |

### Experiment 100

To determine the appropriate CLP mode, the distal 50% of the appendix of male NMRI mice was ligated and perforated with a 0.9 mm cannula 1 time (4 animals), 2 times (4 animals) or 3 times (8 animals). 0 of 8 animals survived three punctures, 3 of 4 animals survived 2 punctures and 2 of 4 animals survived 1 puncture.

### Experiment 101

CLP with 3 punctures was performed on day 0 in 8 male NMRI mice (32-39 g) and (still under ketamine/xyiazine anesthetic) 4 mice were injected i.v. with 45 mg/kg DPOCL in 0.42 ml/10 g KGW, a further 4 mice were injected with an equivalent amount of buffer solution. The injections were well tolerated by the animals. On day 1, as expected, the animals already showed sepsis symptoms and were not to be exposed to further ketamine/xyiazine anesthesia, which (see above) lasts 30 to 60 minutes and cannot be antagonized. Therefore, anesthesia with midazolam, fentanyl and medetomidine was used on day 1, which was then antagonized with flumazenil, atipamezole and naloxone, because there is good experience with this anesthesia for other retrobulbar injections (in healthy mice). The CLP mice tolerated the combination of antagonizable anesthesia and \hat{ }1.5 ml i.v. poorly from day 1 and had to be killed on day 2, regardless of whether they had received DPOCL (4 animals) or PBS (4 animals).

### Experiment 102

Also in this experiment CLP was performed on day 0 with 3 punctures in 8 NMRI mice (28-32 g) and (still under ketamine/xylazine anesthesia) 4 mice were injected i.v. with 45 mg/kg DPOCL in 0.42 ml/10 g KGW, a further 4 mice were injected with an equivalent amount of buffer solution. However, females were used in this experiment and isoflurane was used for anesthesia, as this brief anesthesia should be sufficient for retrobulbar injections. In fact, NMRI females survived CLP with isoflurane better, so that after 1 week 4 out of 4 DPOCL-treated animals were still alive and 2 out of 4 PBS-treated animals. However, even in the females, the high DPOCL volume on day 1, 2 and 3 was associated with some circulatory stress.

### Experiment 103

As in experiment 102, CLP with 3 punctures was performed in 8 male NMRI mice (28-36 g) on day 0 and (still under ketamine/xyiazine anesthesia) 4 mice were injected i.v. with 45 mg/kg DPOCL in 0.42 ml/10 g KGW, a further 4 mice were injected with an equivalent amount of buffer solution. After good experience in experiment 102 isoflurane was again used for the further DPOCL injections. On day 8, 3 out of 4 animals treated with DPOCL and 1 out of 4 after PBS treatment were still alive. The males also had circulation problems due to the high injection volume.

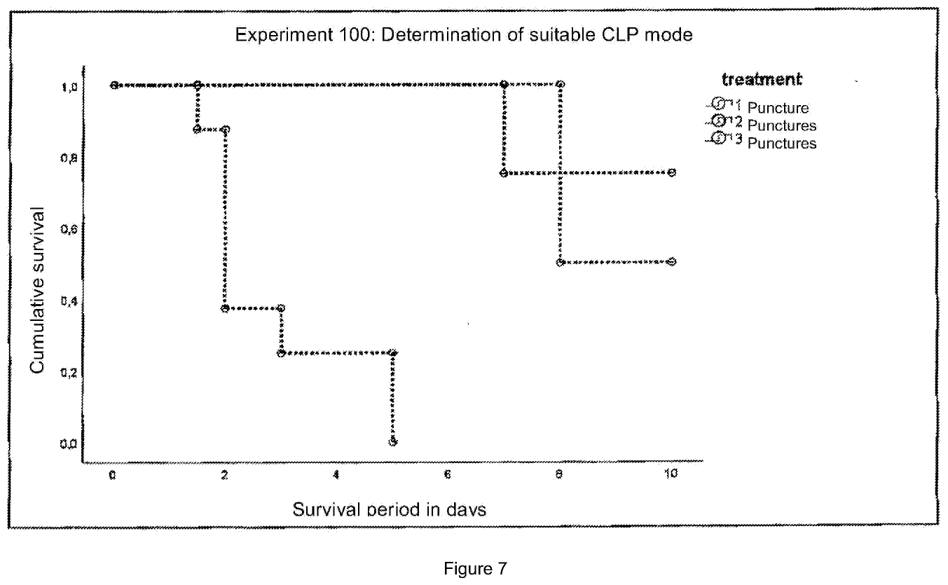

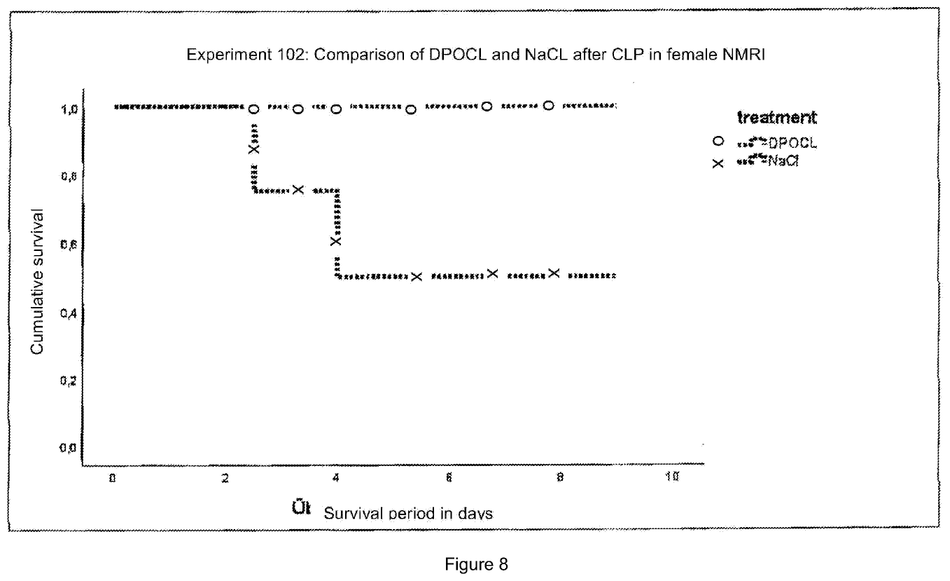

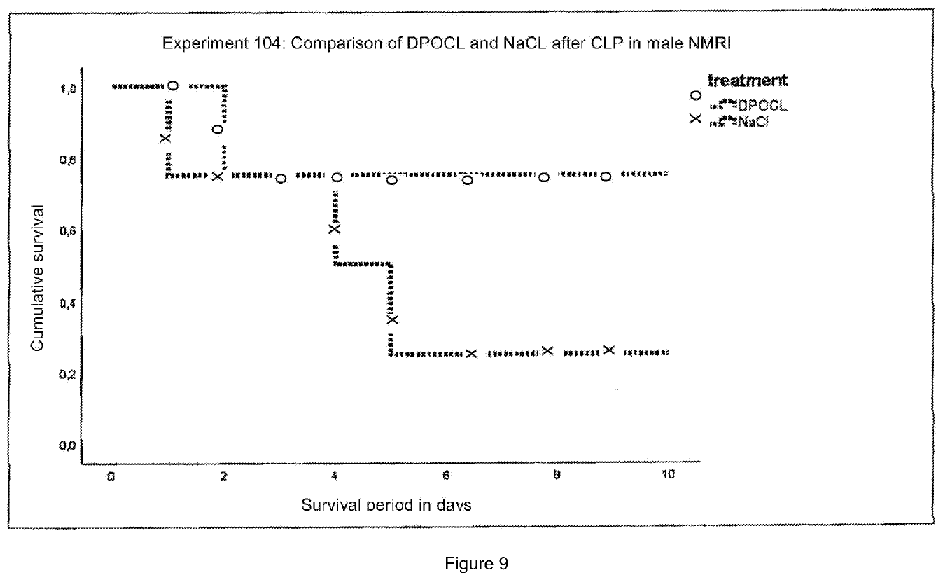

The results of experiments 100, 102 and 103 are represented graphically in FIGS. 7, 8 and 9.

### Experiment 200: Determination of the Minimum DPOCL Dose Required

In experiment 200 further animals are treated with two lower doses of DPOCL after CLP, to determine whether lower doses of DPOCL also protect the animals.

### Group 2.1

18 mice receive directly after CLP 200 μl PBS retrobulbar i.v. on day 0, day 1, day 2 and day 3.

### Group 2.2

18 mice receive directly after CLP 15 mg DPOCL in 200 μl retrobulbar i.v. on day 0, day 1, day 2 and day 3.

### Group 2.3

18 mice receive directly after CLP 5 mg DPOCL in 200 μl retrobulbar i.v. on day 0, day 1, day 2 and day 3.

The survival of the groups is monitored at closer intervals.

### Experiment 300: Determination of the Required Minimum Treatment Period

In experiment 300 the duration of the treatment is shortened by 1 day in each of the different subtrials to determine the minimum time DPOCL needs to be administered for the previously observed improvement of survival to occur.

### Group 3.1

18 mice receive directly after CLP×mg DPOCL in 200 μl retrobulbar i.v. on day 0, day 1, day 2 and day 3.

### Group 3.2

18 mice receive directly after CLP×mg DPOCL in 200 μl retrobulbar i.v. on day 0, day 1, and day 2.

### Group 3.3

18 mice receive directly after CLP×mg DPOCL in 200 μl retrobulbar i.v. on day 0 and day 1.

### Group 3.4

18 mice receive directly after CLP×mg DPOCL in 200 μl retrobulbar i.v. only on day 0.

The survival of the groups is monitored at closer intervals.

Experiment 400: Production of Cytokines and Chemokines (Mediators) 24 h after CLP

As a rule, successful SIRS therapy influences mediator production in the treated animals. Mediators can be determined in different tissues and compartments with and without further stimulation. A mediator measurement in the serum of septic animals 24 h after CLP is often used as a rough guide. IL-6 is determined as the main target variable, other mediators are secondary targets.

In addition, the bacterial load in the blood, peritoneal lavage, lungs, liver and kidneys is a determined as a further secondary target in these animals.

### Group 4.1

8 mice receive 200 μl PBS retrobulbar i.v. directly after CLP on the optimal days determined in experiment 4.

### Group 4.2

8 mice receive directly after CLP the optimal amount of DPOCL in 200 μl retrobulbar i.v. determined in experiment 2 at the optimal time points determined in experiment 3.

### Experiment 500: Use of Evans Blue for Study of Vascular Permeability

A symptom of SIRS is DIC (disseminated intravascular coagulation) in combination with increased vascular permeability. This means that clotting factors and platelets are depleted and are no longer available to close off the endothelial damage that has occurred. The barrier function of the endothelium can be tested by injecting the dye Evans Blue i.v. and 30 min later perfusing the animals, removing organs (lungs, intestines), extracting the dye and detecting it photometrically. The more damage there is to the endothelium, the more dye will penetrate into the tissue.

The biometric design is taken over from experiment 4: group sizes n=8.

### Group 5.1

8 mice receive 200 μl PBS retrobulbar i.v. directly after CLP on the optimal days determined in experiment 4.

### Group 5.2

8 mice receive 200 μl DPOCL retrobulbar i.v. directly after CLP in the optimal amount determined in experiment 3 on the optimal days determine in experiment 4.

48 h after CLP all mice are injected with 200 μl Evans Blue retrobulbar, i.v. 30 min later the mice are anesthetized with ketamine/xylazine and perfused with PBS. The organs are removed and Evans Blue is determined.

The features of the invention disclosed in the above description, as well as in the claims, figures and embodiments, may be essential, both individually and in any combination, for the implementation of the invention in its various embodiments.

1. A pharmaceutical preparation for use in treatment of systemic inflammatory response syndrome (SIRS), containing a reactive chlorine compound as an active ingredient.

2. The pharmaceutical preparation for use according to claim 1, wherein the reactive chlorine compound comprises a peroxochloric acid, a dichloroperoxo acid, and/or a peroxochlorous acid or a pharmaceutically acceptable salt of these acids.

3. The pharmaceutical preparation for use according to claim 1, wherein the reactive chlorine compound comprises a molecular formula selected from the group consisting of HClO₃, HClO₄, and/or H₂Cl₂O₆ or a pharmaceutically acceptable salt of these acids.

4. The pharmaceutical preparation for use according to claim 1, wherein the reactive chlorine compound comprises a structure of formula [O═ClOO]—, [O₂ClOO]—, [O₂ClOOClO₂]²⁻, and/or an anion of the reactive chlorine compound has a molecular formula Cl₂O₆²⁻.

5. The pharmaceutical preparation for use according to claim 1, wherein the reactive chlorine compound is obtained according to a method in which
(a) chlorine dioxide is reacted with an aqueous or water-containing solution of hydrogen peroxide or another hydroperoxide or peroxide at a pH >=6.5,
(b) the pH is lowered to 3 to 6 by addition of an acid, and
(c) a gaseous free reactive chlorine compound is expelled with a cooled gas and collected in a basic solution with a pH >10.

6. The pharmaceutical preparation according to claim 1, wherein the reactive chlorine compound is obtained according to a method in which
(a) chlorine dioxide is reacted with an aqueous or water-containing solution of hydrogen peroxide or another hydroperoxide or peroxide at a pH >=6.5,
(b) the pH is lowered to 3 to 6 by the addition of an acid,
(c) a gaseous free reactive chlorine compound is expelled with a cooled gas and collected in a basic solution with a pH >10, and
(d) the collected reactive chlorine compound is incubated with chlorite at a pH of 6 to 8.

7. The pharmaceutical preparation according to claim 1, comprising a pharmaceutically acceptable carrier.

8. The pharmaceutical preparation according to claim 1, wherein a mass spectrum of the pharmaceutical preparation shows a signal at 189.0 m/z.

9. The pharmaceutical preparation according to claim 1, wherein a mass spectrum of the pharmaceutical preparation shows a signal at 83.2 m/z.

10. The pharmaceutical preparation according to claim 1, wherein a systemic inflammatory response syndrome (SIRS) is associated with a bacterial infection.

11. The pharmaceutical preparation according claim 1, wherein the pharmaceutical preparation contains a pharmaceutically active substance, which differs from the reactive chlorine compound of claim 1.

12. The pharmaceutical preparation according to claim 1, wherein the pharmaceutical preparation is provided in a sachet, which comprises at least two compartments for storage of at least two liquids, which can be opened mechanically, such that, after opening the compartments, the liquids can be mixed, wherein one of the compartments comprises a liquid reactive chlorine compound according to claim 1 and one of the compartments comprises a liquid for adjusting a pH to a physiological pH value.

13. A method of treatment of SIRS in a human subject in need thereof comprising concurrent or sequential administration of hemodialysis treatment and a pharmaceutical preparation according to claim 1 to the subject.

14. A combination preparation comprising separate packages of at least one pharmaceutical preparation according to claim 1 for the treatment of SIRS and at least one drug which differs from the reactive chlorine compound of claim 1.

15. The combination preparation according to claim 14, wherein the drug, which differs from the reactive chlorine compound of claim 1, comprises at least an antibiotic, an antipyretic, a drug for the treatment of disseminated intravascular coagulation (DIC), an antibody, a cytokine, a chemokine, an antimicrobial peptide, a sphingomyelinase inhibitor, a statin, alpha-2-macroglobulin, thrombin-derived C-terminal peptide, sphingosine-1-phosphate, curcumin, ascorbic acid, resveratrol, melatonin, glycyrrhizin, and/or erythropoietin.

16. The pharmaceutical preparation of claim 6, wherein in step (d) the collected reactive chlorine compound is incubated with chlorite at a pH of approximately 7.

17. The pharmaceutical preparation according to claim 7, wherein the pharmaceutically acceptable carrier comprises water, wherein the water content is at least 90 wt. % and the pharmaceutical preparation is an aqueous solution.
